(19) United States
(12) Patent Application Publication
(10) Pub. No.: US 2002/0028213 A1
(43) Pub. Date: Mar. 7, 2002

(54) Vaccines and method therefor

(76) Inventors: Lawrence T. Glickman, West Lafayette, IN (US); Harm Hogenesch, West Lafayette, IN (US)

Correspondence Address: BARNES & THORNBURG, 11 SOUTH MERIDIAN, INDIANAPOLIS, IN, 46204

(21) Appl. No.: 09/933,308

(22) Filed: Aug. 20, 2001

Related U.S. Application Data
(63) Continuation of application No. 09/462,258, filed on Jan. 5, 2000, now abandoned.
Application No. 09/462,258 is a 371 of international application No. PCT/US98/14919, filed on Jul. 17, 1998.
(60) Provisional application No. 60/053,020, filed on Jul. 18, 1997.

(51) Int. Cl. A61K 39/12; C12N 7/00; C12N 7/01; C12N 7/02; C12N 5/00; C12N 5/02

(52) U.S. Cl. 424/204.1; 435/235.1; 435/239; 435/325

(57) ABSTRACT

An improved species specific vaccine having reduced risks of engendering autoimmune disease in vaccinated animals is described. The vaccine is formulated to be substantially free of heterologous species serum components. Preferably the species specific vaccines are produced from antigen concentrates recovered from cell culture media comprising serum wherein the serum consists essentially of homologous species serum.

This application is a continuation of U.S. application Ser. No. 09/462,258, filed Jan. 5, 2000, which is a U.S. national application of international application serial No. PCT/US98/14919 filed Jul. 17, 1998, which claims priority to U.S. provisional application serial No. 60/053,020 filed Jul. 18, 1997, which is expressly incorporated by reference herein.

### FIELD OF THE INVENTION

This invention relates to a method of producing improved vaccines. More specifically this invention is directed to species specific vaccines, particularly antiviral vaccines for humans and companion animals, substantially free of heterologous species serum components and to a method of minimizing the autoimmune side effects of vaccination by use of such vaccines.

### BACKGROUND AND SUMMARY OF THE INVENTION

Autoimmune diseases are a common cause of morbidity and mortality particularly in humans, companion animals such as dogs and cats and other animal species. It is known that humans and animals produce autoantibodies relatively easily when their immune system is appropriately stimulated (Krutitskaya et al., Acta Virologica 40: 173-177, 1996). For example, autoantibodies have been associated with a wide spectrum of clinical syndromes in the dog including skin diseases (pemphigus; vulgaris, pemphigus foliaceous, bullous pemphigoid), hematological diseases (autoimmune hemolytic anemia, autoimmune thrombocytopenia purpura, nonregenerative anemia), muscle diseases (myasthenia gravis, dermatomyositis), rheumatoid arthritis, and systemic lupus erythematosus. Typically the autoantibodies are directed against some very common autoantigens, including DNA, IgG, phospholipids, and erythrocytes.

The various genetic and environmental stimuli that provoke autoantibody production in humans and animals are unknown. However, over the past few years, a growing concern has been expressed by animal owners/breeders and veterinarians regarding adverse health effects associated with use of common vaccines. It has been reported in the recent technical literature that some immune-mediated disorders are exacerbated when vaccinations are administered. Those concerns have been substantiated by recent scientific studies showing a statistical association between immune mediated hemolytic anemia in dogs and vaccination in the previous 30 days. (Duval & Giger, J Vet Int Med, 10:290-295, 1996.) In a thorough examination of medical records for dogs with autoimmune disease, the only factor in common is that the dogs had all received a booster vaccination for distemper and corona virus within the previous 60 days. (Allbritton, Vet Allerg Clin Immunol, 4: 16-17, 1996.)

Vaccination has also been implicated as a trigger for a wide variety of immune-mediated diseases in humans, and these associations have been recently catalogued in a publication from the Centers for Disease Control and Prevention titled “Update: Vaccine Side Effects, Adverse Reactions, Contraindications, and Precautions.” (Centers for Disease Control Prevention, Morb Mort Week Rep 45:(RR12): 1-36, 1996).

Until recently, there was no evidence to link vaccination directly with development of autoantibodies and concomitant immune-mediated disease states in the vertebrate species. An investigation was initiated to determine if commonly used vaccine protocols in dogs resulted in alterations of immune function and increased susceptibility to autoimmune disease. The results indicated that specific autoantibodies developed in vaccinated animals, but not in non-vaccinated animals. Significant concentrations of autoantibodies against laminin, fibronectin, DNA, cardiolipin, collagen, albumin, and transferrin were present in some or all of the vaccinated dogs by 22 weeks of age, at which time the animals were sacrificed for pathological evaluation. A longer-term study confirms that the concentration of autoantibodies that were formed following initial vaccination increased further, following booster vaccinations later in life.

The mechanism by which commercial vaccine induces autoantibody production and related anti-self immune function is still unknown. Vaccines are derived from in vitro cell culture of vaccine antigen propagating cells, for most if not all antiviral vaccines, and typically those vaccine formulations contain contaminants including phylogenetically conserved heterologous cellular and serum antigens. Such contaminant antigens can induce an autoimmune response when injected with the potent adjuvants used in commercial vaccines. The antibodies produced against such conserved vaccine contaminants can cross-react with similar antigens (autoantigens) present in host tissues to engender anti-self immune system function. One primary source of contaminant antigens is the bovine serum commonly used in the culture media that supports the propagation and growth of the vaccine antigen producing cells. The contaminant antigens are not easily separated from the targeted vaccine antigens in current manufacturing processes for animal and human vaccines.

This invention provides a new method for producing effective human and animal vaccines to induce fewer autoimmune responses, even after multiple vaccinations. Vaccine manufacture is carried out in accordance with this invention using cell culture media where the serum component of such media consists essentially of serum prepared from the vaccine targeted species. The resultant species specific vaccines are substantially free of heterologous species serum components that are believed to be at least one significant source of vaccination induced autoantibody production.

Thus the present invention is directed to the production of serum-based species specific vaccines which, when used to immunize the pre-determined animal species, produce fewer side effects manifested by the onset of apparent immune dysfunction and resulting autoimmune disease states in those species. In a preferred embodiment the improvement in the production of vaccines derives from the use of homologous serum, (i.e., serum isolated from the species to be vaccinated) as the culture medium for supporting the growth and proliferation of the antigen producing organism/cells.

### DETAILED DESCRIPTION OF THE INVENTION

The present invention, in general terms, is directed to improved vaccines which can be administered with lower risk of autoantibody production and associated autoimmune disease states consequent to vaccination. The improvement comprises the preparation of vaccine formulations that are substantially free of heterologous serum components. Vaccine formulations prepared in accordance with current procedures contain heterologous serum components that comprise phylogenetically conserved antigens. It has been discovered that under the conditions of vaccination (for example, the use of adjuvants for stimulating antibody production) those heterologous serum contaminant engender the production of antibodies that are cross-reactive with cells and tissues in the vaccinated species. By eliminating the use of heterologous serum in the process for producing vaccine antigens, the risk of inducing autoantibody production in the vaccinated vertebrate species is much reduced.

Therefore, in accordance with one embodiment of the present invention, there is provided a method of minimizing autoimmune side effects associated with vaccination of a warm-blooded vertebrate, particularly vaccination for protection against viral infections. The method comprises the step of inoculating or vaccinating the vertebrate (species) with an effective amount of an antiviral vaccine substantially free of heterologous species serum components. The phrase “heterologous species serum components” is define herein as any component naturally associated with serum isolated from an organism other than the species to be vaccinated.

Because of the difficulty in separating the desired vaccine antigen from the other cellular and acellular components of tissue culture media utilized for propagation of antigen producing cells, the preferred way of eliminating heterologous serum components from vaccines is to eliminate the use of heterologous species' serum in the antigen production process. Most, if not all, culture media utilized for vaccine antigen production include one or more serum components, most typically one of bovine origin. In one preferred embodiment of the present invention, the serum component of the culture medium used for vaccine production is limited to that of the species to which the vaccine is to be administered.

In accordance with one embodiment, a vaccine composition for a predetermined species is provided comprising an antigenic component, a homologous serum component (i.e. a serum component of serum prepared from a member of the predetermined species), and a pharmaceutically acceptable carrier. Typically, the vaccine composition is substantially free of bovine serum components and heterologous serum components and further includes an art-recognized adjuvant. In one embodiment, a serum-based vaccine composition is prepared consisting essentially of an antigenic viral compound (i.e., a viral protein or nucleic acid), an adjuvant, homologous serum components and a pharmaceutically acceptable carrier. The phrase “homologous species serum component” as used herein relates to serum isolated from a member of the same species as the species to be vaccinated. In particular, the term relates to the residual serum components of the culture medium used for vaccine production in accordance with the present invention. Typically the homologous species serum will be isolated from an animal of the same genus, and more preferably the same species as the animal to be vaccinated. In one embodiment, the serum is prepared from the group of vertebrate species consisting of canine, feline, equine, porcine, avian, piscine, and human species, and wherein the immunoglobulin fraction is removed prior to use in the cell culture media.

The present invention is directed particularly to the production of antiviral vaccines for pre-determined vertebrate species including canine, feline, porcine, equine, avian, piscine, and human species. Because the autoimmune related side effects of vaccinations have been particularly problematic in human and companion animal species, (for example canine and feline species, and equine species, receiving multiple lifetime vaccinations) the present invention is particularly significant for vaccine production for such vertebrate species. The present invention can be implemented using art-recognized vaccine production protocols, without modification except for substituting homologous species serum for the common heterologous species serum components of culture media used to propagate antigen-producing organisms. The antigen concentrate recovered from the modified culture media can be used with common pharmaceutically acceptable carriers and art-recognized adjuvants to produce vaccines having reduced risks of vaccination associated immune dysfunction.

In one embodiment of this invention, there is provided a method of producing a serum-based antiviral vaccine for a pre-determined vertebrate species selected from the group consisting of canine, feline, equine, porcine, avian, piscine, and human species. The vaccine comprises a viral antigen capable of evoking systemic production of antibodies to that antigen. The method of preparing the vaccine comprises the steps of propagating an antigen producing organism or cell in a culture medium wherein the serum component of the culture medium consists essentially of the serum from the pre-determined vertebrate species. In accordance with standard antigen producing protocols, the culture medium is processed to recover an antigen concentrate and the resulting antigen concentrate is combined with a pharmaceutically acceptable carrier and optionally with art-recognized adjuvants for improving antigen-antibody titers. By limiting the serum component of the culture medium to serum consisting essentially of serum from the pre-determined species, autoantibody engendering contaminants commonly present in heterologous serum culture medium components, are eliminated from the vaccine formulations. The antigen concentration in such vaccine formulations can be optimized by routine experimentation with the threshold amount that is defined as the amount that is effective in eliciting an antivirally effective antibody titer in the pre-determined vaccinated vertebrate species.

To use homologous serum in culture media for the production of vaccines, the serum needs to be fractionated to remove antibodies present in the serum against the organisms/cells being cultured. Such fractionation procedures are well known in the art, for example, for separating gamma globulin from human serum for the production of Intravenous Gamma Globulin (IVIG). Such separation procedures include cold ethanol fractionation techniques based on procedure of Cohn (Duhem, Clin Exptl Immunol 97(S1) 79-83, 1994). Isolated IVIG is in great demand in human medicine for the treatment of primary immune deficiencies, autoimmune hemolytic anemia, immune thrombocytopenia purpura, and a rapidly growing list of other immune-mediated diseases. A demand for IVIG for other species is anticipated. Canine or feline serum can be fractionated for use in vaccine production using similar procedures; the separated gamma globulin fraction can be used to treat a wide variety of canine or feline immune-mediated diseases. For example, it has been shown human gamma globulin provides an effective treatment for dogs with life-threatening autoimmune hemolytic anemia. Intravenous gamma globulin from dog serum would be as effective and safer than human gamma globulin for treating immune-mediated diseases in the dog, particularly for conditions requiring administration of multiple doses. In accordance with one embodiment, a homologous serum fraction is administered to the predetermined warm blooded vertebrate species to treat immune-related diseases. In one embodiment a substantially pure IVIG fraction is isolated from dog or cat serum, and is administered to the respective dog or cat species to treat immune-mediated diseases such as nonregenerative anemia and autoimmune hemolytic anemia.

### EXPERIMENTAL RESULTS

The relationship between vaccination and production of autoantibodies has been evaluated in dogs in both a short and a long term study. The long term study is ongoing. In the short term study lasting twenty-two weeks, two groups of five dogs were examined—a control group and a test group. The test group was vaccinated at 8, 10, 12, 16 and 20 weeks with the VANGUARD 5/cv-1 (Pfizer) vaccine and at 16 weeks with a rabies vaccine (IMRAB-3, Rhone-Merieux). The control group received sham immunizations of saline solution using the same schedule as for the treatment group.

All the animals in the short term study were monitored daily for the presence of clinical and behavioral abnormalities, and their body weight and temperature were measured weekly. Blood samples were collected for a variety of tests including blood chemistry, CBC, flow cytometry, DNA isolation for RFLP analysis, hormone levels, viral titers, serum immunoglobulin, and autoantibody determinations.

Serum samples from the dogs were collected at the beginning of the study and were screened by enzyme-linked immunoabsorbent assay (ELISA) for reactivity against an antigen panel containing 16 compounds (six autoantigens and 10 homologous antigens) which included antigens for which autoantibodies had been reported in various human and animal autoimmune related processes. The broad antigenic panel allowed the identification of appropriate markers for vaccine-induced immunological changes.

Serum IgG reactivity estimated by indirect ELISA varied within antigens, with absorbance values at 405 nm (A₄₀₅) ranging from 0.1 to greater than 3.0. High sera reactivity (A₄₀₅ greater than 0.7) was detected against laminin fibronectin, and skeletal muscle myoglobin and myosin. The majority of the remaining antigens tested, revealed moderate levels of reactivity (A₄₀₅, 0.2-0.4). Statistically significant differences were detected between the vaccinated and unvaccinated groups at the end of the study for canine (albumin, and transferrin); bovine (cardiolipin, DNA, fibronectin, and sphingomyelin) and murine (collagen type IV and laminin) antigens.

Serum IgG of three animals in the treatment group had high reactivity (A₄₀₅ between 0.8 and 2.0) against murine laminin, while the other two vaccinated dogs and the five unvaccinated dogs had negligible levels of reactivity. Serum samples collected at different times following vaccination were tested in the reactive animals. The presence of antilaminin antibodies in only some of the vaccinated dogs, and the different kinetics suggest genetically determined predisposition to autoimmunity.

All animals in the vaccinated group (T108, T126, T321, T772, & T853) had very high levels of antibodies to bovine fibronectin (A₄₀₅ values from 2.0 to 3.5). (See FIG. 1 +L). Three dogs (T108, T126, & T853) begin to show serum fibronectin antibodies following the second vaccination; they reached peak levels after the third vaccination and remained fairly constant until after the end of the study. The two other animals reached comparable levels of anti-fibronectin antibodies after the fourth and fifth vaccination period. (See FIG. 2 +L., the timing of the five vaccinations is indicated by arrows) Very high levels of anti-fibronectin antibodies were also found when the samples were assayed against human and murine plasma fibronectin. The strong response against fibronectin in all vaccinated animals reflects the presence of bovine fibronectin, a normal constituent of serum, in the vaccine preparations. Bovine serum is commonly used as a growth supplement for the cells in which viruses are cultured for vaccine manufacture.

Moderate levels of serum reactivity (significantly increased in comparison to the control group) against other antigens, for example, DNA (bovine) and cardiolipin (bovine) was found in vaccinated animals. These autoantibodies are associated with various autoimmune phenomena, including systemic lupus erythematosus, idiopathic thrombocytopenia, and reproductive disorders. Lower, but significant levels of reactivity against canine albumin and transferrin were also found.

Laminin, collagen type IV, and fibronectin are extracellular matrix proteins ubiquitously present in basement membranes. In the past decade, there has been an interest in the immunological potential of basement membrane components. In particular the presence of elevated levels of antibodies against laminin and collagen type IV have been reported in various systemic autoimmune processes, including lupus, erythematosus, rheumatoid arthritis, vasculitis, and cardiomyopathy. Few studies have reported on the presence of anti-fibronectin antibodies in autoimmune disease, but their presence has been noted in human patients with systematic lupus erythematosus. Thus, the finding of the large amounts of serum anti-fibronectin and antilaminin antibodies in the vaccinated animals, together with moderate levels of anticollagen type IV antibodies is of clinical significance to the dog autoimmune phenomena related to intensive vaccinations at early stages of life.

Increased levels of antibodies reactive to canine skeletal muscle myoglobin and antiporcine skeletal muscle myosin were present in both the vaccinated and unvaccinated animals at the end of the study. There was no statistically significant differences between both groups, although there were significant differences between the starting point and the end point levels in both groups. Analysis of these antibodies over time revealed increased levels of antibodies reactive with skeletal myoglobin and myosin in both groups after the second injection.

An analysis of the data reveals a significant change in the antibody repertoire of the vaccinated dogs. There are several possible explanations for this change in the antibody repertoire: (1) The vaccines contain highly conserved cellular and serum antigens. In the presence of potent adjuvants, which are included in the vaccines, these components may induce an immune response, which cross-reacts with autoantigens. This mechanism may be similar to the way in which autoimmunity is commonly induced in experimental animals by injecting cross-reactive antigens with an adjuvant. This is the most likely explanation for the high level of antigen fibronectin antibodies in the vaccinated animals. (2) Antibodies to vaccine-components or antiidiotipic antibodies cross-react with epitopes expressed on auto antigens. (3) In the process of immune response, B and T cells that are not specific for the components of the vaccine may become activated as “bystanders”. It has been estimated that only 10 to 20% of activated lymphocytes express receptors specific for the antigen to which the immune response was induced. In addition, the specificity of antibodies may be altered as a result of somatic mutation in the variable region. This mechanism has been invoked to explain the occurrence of anti-DNA antibodies after immunization of mice with an unrelated antigen. (4) Finally, the immune system is regarded by certain immunologists as a network of interconnected antibody and T cell reactivities. The immune response against a foreign antigen is seen as a disturbance of this network. In a young animal this network is still being formed and external influences may have a great impact on the formation of the network and the resulting antibody and T-cell repertoire.

Serum antibodies against a variety of antigens are detected at variable levels in animals vaccinated with a commercially available multivalent vaccine. High levels of anti-laminin and anti-fibronectin antibodies were found while moderate and low levels of serum antibodies were observed against other antigens, including DNA, cardiolipin, collagen, and albumin transferrin.

In the ongoing long-term study, twenty purebred Beagle dogs from five litters were divided into four groups (A, B, C, and D) so as to be comparable in terms of body weight and litter of origin. The five dogs in the group “A” are immunized at 8, 10, 12, 16, 20, 26, and 52 weeks of age with a multivalent commercial vaccine (Pfizer, VANGUARD 5/CV-L®) containing attenuated canine viral strains of distemper, adenovirus type 2, parainfluenza, and parvovirus, an inactivated strain of canine corona virus, and inactivated whole culture of Leptospira canicola and L. icterohaemorrhagiae. They also receive a rabies vaccine (IMRAB®3 (killed virus), Rhone-Merieux,) at 16 and 52 weeks of age. Animals in group “B” are only immunized with the VANGUARD 5/CV-L vaccine following the same schedule described for group “A”. Dogs in group “C” just receive the rabies vaccine (as in group “A”), while the animals of group “D” are given sham immunizations of saline solution using the same schedule as in group “A”.

As in the shorter study, the animals are monitored for the presence of clinical and behavior abnormalities including general health observations, temperature, body weight, CBC and blood chemistry. All such observations to date have been within normal limits.

Blood samples were collected at 8 and 16 weeks and examined for antibodies to distemper and parvovirus. The animals in groups “A” and “B” which were immunized with the multivalent vaccine have sero converted. All dogs in the “C” and “D” group remained seronegative. A preliminary assessment of the presence of anti-laminin antibodies in all twenty dogs has been done for serum samples which were collected at 18 weeks of age. In one animal from group “A”, the serum anti-laminin antibody was significantly elevated in comparison to antibody level in the animals of the same age in the previous short term study. While any conclusions at this stage of the project are still premature, the preliminary results concur with the results attained in the short term study.

### GENERAL VACCINE PREPARATION/TECHNIQUES

A general procedure for vaccine preparation requires the preliminary step of isolating a suitable antigen propagating cell population. One example is canine kidney cells. Canine kidneys are processed, and the resulting cells are cultivated as a monolayer on the surface of a glass bottle which contains a standard culture medium in accordance with well-known and customary procedures. A good culture medium for starting the growth of the cells is referred to as Eagles MEM (minimum essential medium). This medium is typically supplemented with serum and antibiotics. At first, there are several different kinds of cells growing together, such as the fibroblasts from connective tissue and the kidney tubular epithelial cells. The cells may also be contaminated with a virus peculiar to the host animal; for example, in the instance of canine cells, the cells may be contaminated with some latent viruses as canine distemper and infectious canine hepatitis. To avoid these latent viruses fetal canine tissues may be utilized. The cells multiply in the bottle until a monolayer formation is complete. The cell population may be further increased by transferring portions of them to other bottles. This is accomplished by treating them with trypsin to remove them from the glass surface and dividing them into other bottles with fresh culture medium. When the required number of bottles have been prepared and the cells have stopped growing, it is customary to add maintenance medium such as a mixture of Earl's salt solution, lactalbumin hydrolysate, a reduced amount of serum, and antibiotic. The above procedure is conventional practice, and it is important for optimum production to start vaccine production before the cells start to degenerate; consequently, it has been necessary to provide fresh cell cultures in making each new serial lot of cells.

An alternative to the above-described technique of vaccine production is the use of established cell lines, formerly referred to as stable cell lines. The technique of applying stable cell lines for virus production is also well known in the art.

An established cell line is derived by the serial subculture of cells from normal tissue growing in tissue culture until it can grow perpetually. It contains only one type of cell and every cell is genetically related. Because of this relationship, each individual cell, within the limits of biological variance, has the same biochemical characteristics, the same growth rate, and the same virus susceptibility, and, when infected with a virus, will respond in the same manner.

Once the antigen has been isolated from the appropriate tissue culture, the antigen must be made to be non-pathogenic to the targeted species. Antigens may be made non-pathogenic by any of the methods known in the art. There are three types of vaccines which are utilized to produce active immunity. One is made from whole, inactivated or killed pathogens or components of these pathogens. Another is a toxoid. Toxoids are antigens which have been treated either physically or chemically so that they no longer produce clinical diseases. Such treatment includes utilizing any number of a wide variety of art-recognized inactivating agents including, but not limited to, binary ethyleneimine, acetyl ethyleneimine, beta-proprio lactone, formalin, formaldehyde, phenol, ultra-violet radiation or gamma radiation. A third vaccine type is an attenuated vaccine. Such vaccines are prepared from antigens which have undergone repeated passages through laboratory cultures; they remain infectious but lose the ability to induce clinical disease. The present invention offers advantage in the preparation of each of such vaccine types.

Antiviral vaccine preparation typically requires the use of cell cultures. Different cell lines are used for different viruses, but all cell lines require serum to support their maintenance and growth. Bovine serum is usually used because of its availability and convention. Thus viral vaccines commonly contain bovine serum components that may cause allergic and autoimmune reactions. Attempts have been made to reduce the amount serum necessary for cell culture, but complete elimination is generally not possible. (McKeehan, et al., Develop. Biol. Standard. 37: 97-108, 1977.) The present invention is directed to the use of homologous serum instead of bovine serum, to reduce the potential for autoimmune and allergic reactions in vaccinates. The protocol is generic and has been modified from published protocols. (Duffy, J. I. Noyes Data Corp. Park Ridge, N.J., 1980, pp. 70-111.) Specific details will vary for different viruses and cell culture types. Homologous Serum will be pooled from a minimum of 10 animals or human beings depending on the type of vaccine, e.g., canine serum will be used for the production of vaccines for use in dogs. The immunoglobulin fraction of the serum will be removed by cold-ethanol fractionation (removal of Cohn's fraction II). The remaining serum fraction will be referred to below as homologous serum. Alternatively, whole serum can be used from specific pathogen free animals, i.e., animals of the same species that do not have serum antibodies against the specific virus in the vaccine being produced (also referred to as homologous serum).

Cells (appropriate for the particular virus) are cultured in medium with 5-10% homologous serum at 37° C. until a monolayer is produced. The medium is replaced with fresh medium containing 2% homologous serum, and inoculated with virus. After several days (number depends on the particular virus), the cultures are frozen and thawed to release the intracellular virus. The medium with virus is harvested, lyophilized, and aliquotted in vaccine doses.

The vaccine may be administered by any of the methods known in the art. This includes the use of an adjuvants. Individual or combinations of various adjuvants may be utilized to enhance the immune response. Adjuvants including, but not limited to, aluminum phosphate, ethylene maleic anhydride, neocryl A640, aluminum hydroxide, and oil in water suspensions may be utilized.

Various methods for vaccine preparations have become standardized and well known for those practicing in the art. The following examples of vaccine preparations are meant to be illustrative in nature and provide general examples for the present invention of producing vaccines utilizing homologous serum. Practitioners skilled in the art of vaccine preparation will recognize that homologous serum can be substituted in a variety of methods for vaccine preparation. The following examples are illustrative only and are not intended to reduce the scope of the present invention.

### EXAMPLE 1

A virus strain for canine Corona vaccine is isolated from a dog which died of the CCV enteritis. The virus is serially propagated in crandal feline kidney tissue (CRFK) culture. This virus is passaged several times through CRFK culture to produce the attenuated modified live CCV vaccine. Other attenuated modified live CCV vaccines may be used. The cell cultures are grown in Eagles minimal essential medium (MEM) supplemented with vitamins, non-essential amino acids, sodium pyruvate, sodium bicarbonate, and L-glutamine; finally, Gentamicin (30 mcg/ml) is added as a preservative. A 5 to 10% concentration of canine serum is added for cell growth. The serum concentrate is reduced to 1% for maintenance medium. Trypsin is added at a concentration of 0.5 mls per liter of medium to promote virus infectivity. Cultures of CRFK cells are trypsinized and grown at 20° to 40° C. Sufficient virus is added to achieve a minimum multiplicity of infection ratio of 1:100. The virus is allowed to absorb on monolayers or in suspension for two hours at 35° to 38° C. The resulting fluids are harvested along with the cellular material 48 hours after infection, dispensed and frozen at −48° C. or lower. The material is thawed and blended with medium and SPGA (sucrose, phosphate, glutamate, albumin) stabilizer and lyophilized.

### EXAMPLE 2

The preparation of parainfluenza virus Type I can be accomplished using homologous serum. Isolation and adaption of parainfluenza Types I and III can be accomplished by at least one and preferably at least from three to five passages in monkey kidney cell culture. Isolation and adaption of the parainfluenza virus Type II can be accomplished by at least one and preferably by at least three to five passages from monkey kidney cell culture followed by at least one or preferably from three to five passages from embryonated hens' eggs, preferably via the amniotic route. The parainfluenza virus is added to a glass bottle containing chick embryo tissues prepared from minced and trypsinized, approximately 10-day old chick embryos for Types II or III, or for Type I human diploid lung fibroblasts such as WI-38 fibroblasts as described in Exper. Cell. Res., 25, 585 (1961). The culture medium may be selected from any of those that support cell growth and may be, for example, the known medium Eagles essential medium (MEM) in combination with Eagles balanced salt solution (BSS) supplemented with homologous serum. After the addition of the virus, the infected cell cultures are incubated in successive passages at 30° to 38° C. During these passages the virus is replicated in large amounts and becomes attenuated.

Neomycin is incorporated into the growth and maintenance medium. Multiple harvests of the virus are collected at 2-4 day intervals and the bottle cultures are refed with fresh maintenance medium containing stabilizer. A viral stabilizer, for example sucrose, phosphate, or glutamate, is added in appropriate amounts to the virus harvests prior to freezing and storage at −78° C. One or more appropriate harvests are selected following completion of infectivity titrations. The collected fluids are lyophilized. Following the lyophilization, the virus is stored in vials which are capped, sealed, and retained for reconstitution as a vaccine by the addition of sterile water or an appropriate adjuvant.

1. A method of producing an antiviral vaccine for a predetermined vertebrate species selected from the group consisting of canine, feline, equine, porcine, avian, piscine, and human species, said vaccine comprising an amount of viral antigen effective in evoking systemic production of antibodies to said antigen, said method comprising the steps of:
propagating an antigen producing cell in a culture medium, said culture medium comprising serum wherein the serum consists essentially of serum from said predetermined vertebrate species;
recovering an antigen concentrate from said culture medium; and,
combining said antigen concentrate with a pharmaceutically acceptable carrier.

2. A vaccine prepared in accordance with the method of claim 1.

3. A feline vaccine prepared in accordance with the method of claim 1.

4. A human vaccine prepared in accordance with the method of claim 1.

5. A canine vaccine prepared in accordance with the method of claim 1.

6. An improved antiviral vaccine for a predetermined vertebrate species, said vaccine comprising
a homologous serum component;
an antigenic component, the antigenic component produced in a culture comprising the homologous serum component; and
a pharmaceutically acceptable carrier, wherein said composition is substantially free of bovine serum components and heterologous serum components.

7. The vaccine of claim 6 wherein the antigenic component comprises viral proteins.

8. The vaccine of claim 6 further comprising an adjuvant.

9. In a method for producing an antiviral vaccine for a predetermined vertebrate species consisting of canine, feline, equine, porcine, avian, piscine, and human species wherein an antigen concentrate is isolated from a culture medium used to propagate an antigen producing organism or cell, said culture medium comprising, serum and wherein the antigen concentrate is used to formulate said vaccine, the improvement which comprises using a culture medium wherein the serum component consists essentially of serum from the predetermined species.

10. A vaccine prepared in accordance with the improved method of claim 9.

11. A method for vaccinating a predetermined vertebrate species with an antiviral vaccine said method comprising the step of administering a vaccine prepared in accordance with the method of claim 1 in an amount effective to elicit an antivirally effective antibody titer in said predetermined vertebrate species.

12. The method of claim 11 wherein the pre-determined species is human.

13. The method of claim 11 wherein the pre-determined species is canine.

14. The method of claim 11 wherein the pre-determined species is feline.

15. A method for vaccinating a predetermined vertebrate species with an antiviral vaccine said method comprising the step of administering a vaccine prepared in accordance with the improved method of claim 9 in an amount effective to elicit an antivirally effective antibody titer in said predetermined vertebrate species.

16. A method of minimizing autoimmune side effects associated with vaccination of a warm-blooded vertebrate selected from canine, feline, equine, porcine, avian, piscine, and human species for protection against viral infection, said method comprising the steps of inoculating said vertebrate with an effective amount of an antiviral vaccine substantially free of heterologous species serum components.

17. The method of claim 16 wherein the antiviral vaccine is produced by
propagating an antigen producing cell in a culture medium, said culture medium comprising serum wherein the serum consists essentially of serum from said predetermined vertebrate species;
recovering an antigen concentrate from said culture medium; and,
combining said antigen concentrate with a pharmaceutically acceptable carrier.

18. The method of claim 16 wherein the warm-blooded vertebrate is a canine species.
